(19) United States
(12) Patent Application Publication
(10) Pub. No.: US 2018/0011101 A1
(43) Pub. Date: Jan. 11, 2018

(54) MATERIALS AND METHODS FOR DETECTING ANDROGEN RECEPTOR SPLICE VARIANTS AND USES THEREOF

(71) Applicants: Ventana Medical Systems, Inc., Tucson, AZ (US); University of Washington, Seattle, WA (US)

(72) Inventors: Alisa Tubbs, Phoenix, AZ (US); Yixin Wang, Basking Ridge, NJ (US); Lawrence D. True, Seattle, WA (US)

(21) Appl. No.: 15/706,577

(22) Filed: Sep. 15, 2017

Related U.S. Application Data
(63) Continuation of application No. PCT/EP2016/055632, filed on Mar. 16, 2016.
(60) Provisional application No. 62/133,763, filed on Mar. 16, 2015.

(51) Int. Cl. G01N 33/574

(52) U.S. Cl. CPC G01N 33/57434; G01N 2333/723; G01N 2800/52

(57) ABSTRACT

The present disclosure relates to materials and methods for evaluating prostate cancer using binding entities (such as antibodies) that bind to the N-terminus and the C-Terminus of androgen receptor. Prostate samples are histochemically labeled for the N-terminus and the C-Terminus of androgen receptor, and a ratio between the binding of the N- and C-terminal antibodies is determined.

### CROSS-REFERENCE TO RELATED APPLICATIONS

This is a continuation of PCT/EP2016/055632, filed Mar. 16, 2016, which claims the benefit of U.S. Provisional Patent Application No. 62/133,763, filed Mar. 16, 2015, the content of each of which is herein incorporated by reference in its entirety.

### BACKGROUND OF THE SUBJECT DISCLOSURE

### I. Field of the Subject Disclosure

The present applications relates to materials and methods for performing diagnostic assays, particularly in the field of prostate cancer diagnostics.

### II. Brief Description of Related Art

Male hormone signaling acting through the Androgen Receptor (AR) plays a dominant role in prostate cancer development, and the development of Castrate-Resistant Prostate Cancer (CRPC) is commonly associated with an aberrant ligand-independent activation of AR. Recent findings suggest that expression of certain AR splice variants in prostate cancer cells is correlated with prognosis and resistance to AR targeted therapies. See, e.g., Zengerling, et al., Effects of sorafenib on C-terminally truncated androgen receptor variants in human prostate cancer cells., Int J Mol Sci. 13:11530-11542 (2012); Mostaghel, et al., Resistance to CYP17A1 inhibition with abiraterone in castration-resistant prostate cancer: induction of steroidogenesis and androgen receptor splice variants, Clin Cancer Res. 17(18):5913-25 (Sep. 15, 2011); Harada, et al., Androgen deprivation causes truncation of the C-terminal region of androgen receptor in human prostate cancer LNCaP cells, Cancer Sci. Vol. 103, Issue 6, pp. 1022-27 (June 2012); Zhang, et al., Androgen receptor variants occur frequently in castration resistant prostate cancer metastases, PLoS ONE, 6:e27970 (2011); Thadani-Mulero, et al., Androgen receptor splice variants determine taxane sensitivity in prostate cancer, Cancer Res. 74:2270-2282 (2014); Antonarakis, et al., AR-V7 and resistance to enzalutamide and abiraterone in prostate cancer, N Engl J Med. 371:1028-1038 (2014). However, to our knowledge, nobody has successfully developed an assay that can predict which patients that express one of these variant splice forms is likely to be resistant to an AR-targeted therapy and/or a chemotherapeutic.

### SUMMARY OF THE SUBJECT DISCLOSURE

The present disclosure relates generally to methods using a combination of two binding entities to detect C-terminal splice variants of androgen receptor (AR) in prostate cancer: (1) a binding entity that binds specifically to an epitope in the N-terminal portion of AR, which should bind to both full-length and C-terminal truncated forms of AR; and (2) a binding entity that binds specifically to an epitope in the C-terminal ligand binding domain of AR, which should only bind to full-length androgen receptor.

In an aspect, a method of tracking progression of a prostate cancer in a patient is provided, the method comprising: (a) microscopically detecting binding of a first binding entity and a second binding entity to a prostate tumor sample from the patient, wherein the first binding entity binds specifically to the N-terminal domain of androgen receptor (AR) and the second binding entity binds specifically to the C-terminal ligand binding domain of AR; (b) calculating a ratio of B₁/B₂, wherein: B₁ is binding of the first binding entity to the prostate tumor sample, and B₂ is binding of the second binding entity to the prostate tumor sample; and (c) comparing the ratio of (b) to a reference ratio calculated according to (b) for a sample of the same tumor taken from the patient at an earlier time point; wherein an increase in the ratio compared to the reference ratio indicates progression of the prostate cancer.

In another aspect, a method of prognosing a prostate cancer in a patient is provided, the method comprising: (a) microscopically detecting binding of a first binding entity and a second binding entity to a prostate tumor sample from the patient, wherein the first binding entity binds specifically to the N-terminal domain of androgen receptor (AR) and the second binding entity binds specifically to the C-terminal ligand binding domain of AR; (b) calculating a ratio of B₁/B₂, wherein: B₁ is binding of the first binding entity to the prostate tumor sample, and B₂ is binding of the second binding entity to the prostate tumor sample; and (c) comparing the ratio of (b) to a reference ratio; wherein a higher ratio of (b) compared to the reference ratio indicates a poor prognosis. In an embodiment, the reference ratio is a ratio calculated according to (b) for a sample of the same tumor taken from the patient at an earlier time point. In another embodiment, the reference ratio is a ratio calculated according to (b) for a representative number of prostate tumors taken from a general patient population. As an example, the reference ratio may represent a cutoff separating patients expected to respond to an AR-targeted therapeutic agent or a chemotherapeutic agent from patients expected to be resistant to the same AR-targeted therapeutic agent and/or chemotherapeutic agent. In an embodiment, the AR-directed therapeutic agent is an AR antagonist or an inhibitor of androgen synthesis. In another embodiment, the AR-targeted therapeutic agent is selected from the group consisting of abiraterone, enzalutamide, Orteronel, Galeterone, ARN-509, ODM-201, AZD3514, EZN-4176, and BMS-641988. In another embodiment, the chemotherapeutic agent is a taxane, such as paclitaxel or docetaxel.

In another aspect, a method of predicting resistance of a patient having prostate cancer to an androgen receptor (AR)-targeted therapeutic agent and/or a chemotherapeutic agent is provided, the method comprising: (a) microscopically detecting binding of a first binding entity and a second binding entity to a prostate tumor sample from the patient, wherein the first binding entity binds specifically to the N-terminal domain of androgen receptor and the second binding entity binds specifically to the C-terminal ligand binding domain of androgen receptor; (b) calculating a ratio of B₁/B₂, wherein: B₁ is binding of the first binding entity to the prostate tumor sample, and B₂ is binding of the second binding entity to the prostate tumor sample; and (c) comparing the ratio of (b) to a reference ratio; wherein a higher ratio according to (b) as compared to the reference ratio indicates that the prostate cancer is unlikely to respond to the AR-targeted therapeutic agent and/or a chemotherapeutic agent. In an embodiment, the reference ratio is a ratio calculated according to (b) for a sample of the same tumor taken from the patient at an earlier time point. In another embodiment, the reference ratio is a ratio calculated according to (b) for a representative number of prostate tumors taken from a general patient population. In an embodiment, the AR-directed therapeutic agent is an AR antagonist or an inhibitor of androgen synthesis. In another embodiment, the AR-targeted therapeutic agent is selected from the group consisting of abiraterone, enzalutamide, Orteronel, Galeterone, ARN-509, ODM-201, AZD3514, EZN-4176, and BMS-641988. In another embodiment, the chemotherapeutic agent is a taxane, such as paclitaxel or docetaxel.

In another aspect, a method of selecting a treatment course for a patient having prostate cancer is provided, the method comprising: (a) characterizing the prostate cancer for the presence of C-terminal deletion splice variants of androgen receptor by: (a1) microscopically detecting binding of a first binding entity and a second binding entity to a prostate tumor sample from the patient, wherein the first binding entity binds specifically to the N-terminal domain of androgen receptor and the second binding entity binds specifically to the C-terminal ligand binding domain of androgen receptor; (a2) calculating a ratio of B₁/B₂, wherein: B₁ is binding of the first binding entity to the prostate tumor sample, and B₂ is binding of the second binding entity to the prostate tumor sample, and (a3) comparing the ratio of (a2) to a reference ratio; and (b) selecting the treatment course based on (a), wherein: (b1) an aggressive treatment course is selected when the ratio of (a2) is greater than the reference ratio; and (b2) a conservative treatment course is selected when the ratio of (a2) is less than the reference ratio. In an embodiment, the aggressive treatment course comprises surgical removal of the tumor and/or prostate, castration, and/or radiation therapy and the conservative treatment course comprises surveillance or administration of an AR-directed therapeutic agent and/or a chemotherapeutic agent. In an embodiment, the AR-directed therapeutic agent is an AR antagonist or an inhibitor of androgen synthesis. In another embodiment, the AR-targeted therapeutic agent is selected from the group consisting of abiraterone, enzalutamide, Orteronel, Galeterone, ARN-509, ODM-201, AZD3514, EZN-4176, and BMS-641988. In another embodiment, the chemotherapeutic agent is a taxane, such as paclitaxel or docetaxel.

In an embodiment, a method of monitoring a treatment course of a prostate cancer in a patient is provided, the method comprising: (a) microscopically detecting binding of a first binding entity and a second binding entity to a prostate tumor sample from the patient, wherein the first binding entity binds specifically to the N-terminal domain of androgen receptor (AR) and the second binding entity binds specifically to the C-terminal ligand binding domain of AR; (b) calculating a ratio of ratio of B₁/B₂ at a plurality of time points during a treatment course, wherein: B₁ is binding of the first binding entity to the prostate tumor sample, and B₂ is binding of the second binding entity to the prostate tumor sample; and (c) comparing the ratios of (b); wherein an increase in the ratio during the course of treatment indicates progression of the prostate cancer and/or resistance to the treatment course. In an embodiment, the treatment course comprises surveillance or administration of an AR-directed therapeutic agent and/or a chemotherapeutic agent. In an embodiment, the AR-directed therapeutic agent is an AR antagonist or an inhibitor of androgen synthesis. In another embodiment, the AR-targeted therapeutic agent is selected from the group consisting of abiraterone, enzalutamide, Orteronel, Galeterone, ARN-509, ODM-201, AZD3514, EZN-4176, and BMS-641988. In another embodiment, the chemotherapeutic agent is a taxane, such as paclitaxel or docetaxel. In another embodiment, a further treatment course is selected when the ratio increases, the further treatment course comprising surgical removal of the tumor and/or prostate, castration, and/or radiation therapy and optionally repeating (a) and (b) during the course of the further treatment, wherein the further treatment is halted if the ratio increases during the course of the further treatment.

In any of the foregoing methods, binding of the first and second binding agent may detected histochemically or cytochemically, preferably immunohistochemically. In an embodiment, the first binding entity and/or second binding entity is an antibody. In one specific embodiment, the first binding T entity is monoclonal antibody SP107 or an antibody that competes with SP107 for binding to androgen receptor and/or the second binding entity is monoclonal antibody SP242 or an antibody that competes with SP242 for binding to androgen receptor. The first binding entity and the second binding entity may labeled with a chromogenic agent or a fluorescent agent. In one specific embodiment, the chromogenic agent or the fluorescent agent is attached to a third binding entity capable of specifically binding to the first binding entity and a fourth binding entity capable of binding to the second binding entity. In another specific embodiment, the first binding entity and the second binding entity comprise a non-endogenous hapten and the third binding entity and the fourth binding entity are antibodies or fragments thereof capable of specifically binding to the non-endogenous hapten. In another specific embodiment, the third binding entity and the fourth binding entity are anti-species antibodies.

In an embodiment, B₁ and B₂ are measures of signal intensity. For example, in one embodiment, B₁ and B₂ are H-scores calculated for the first and second binding agents and the ratio of B₁:B₂ is a ratio of the H-scores. The H-scores preferably are automatically calculated using a digital image of the sample of tumor tissue. In one example, the H-score is automatically calculated using a positive pixel elective algorithm. In an embodiment, the H-scores are calculated on the basis of nuclear and cytosolic staining of the first and second binding agents. In another embodiment, the H-scores are calculated on the basis of staining intensity of the first and second binding agents.

### BRIEF DESCRIPTION OF THE DRAWINGS

FIG. 1 is an image of an RT-PCR used to ensure that the cell lines used express the expected AR or AR variant in LNCaP, VCaP, CWR22v1, and PC3 cell lines.

FIG. 2 is an image of a Western Blot used to ensure that the cell lines used express the expected AR or AR variant in LNCaP, VCaP, CWR22v1, and PC3 cell lines.

FIG. 3 is an image of an immunohistochemical stain of LNCaP, VCaP, CWR22Rv1, and PC3 cell lines using the SP107 and SP242 antibodies.

FIG. 4 is a bar graph of H-scores from the immunohistochemical stain shown in FIG. 3.

FIG. 5 is an image of an IHC staining on cell lines transfected with full length AR (M12 AR) or AR C-terminal splice variant ARv7 (M12 ARv7), primary prostate tumors (PCa), and metastatic tumors (CRPC) using SP107 and SP242.

FIG. 6 is a bar graph of H-scores calculated from the images of FIG. 5.

FIG. 7 is a bar graph of the ratio of SP107 and SP242 H-scores from FIG. 6 for each sample.

### DETAILED DESCRIPTION OF THE SUBJECT DISCLOSURE

The present methods relate to methods for histochemically or immunohistochemically assaying, evaluating, and scoring androgen receptor expression in prostate tumor samples by labeling prostate tumor tissue with a combination of two specific binding entities: (1) a binding entity specific for the N-terminal portion of AR, which binds to both full-length and C-terminal truncated forms of AR; and (2) a binding entity specific for the C-terminal ligand binding domain of AR, which binds only to full-length AR. Binding of the two binding entities is detected and is represented as the quantity B₁ and B₂, respectively. A ratio between B₁ and B₂ is then calculated, which can be used to tracking progression of a prostate cancer, prognose a prostate cancer, predict resistance to an androgen receptor (AR)-targeted therapeutic agent and/or a chemotherapeutic agent, select a treatment course for a patient having prostate cancer, and/or monitor a treatment course of a prostate cancer.

### I. Prostate Tumor Samples

Samples useful in the disclosed process will generally be prostate tumor samples from a prostate cancer patient. As used herein, a “prostate cancer patient” refers to a subject or individual diagnosed with or suspected of having prostate cancer. As used herein, a “subject” or “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. As used herein, the term “prostate tumor sample” shall refer to any samples from the patient containing tumor cells of prostate origin, including samples obtained from primary tumors, metastatic tissue, and circulating tumor cells. Exemplary prostate tumor samples include: solid prostate tumor samples, such as core biopsies or resections; dissociated cell samples from a prostate tumor, such as a fine needle aspirate; and samples of circulating tumor cells of prostate origin, such as those obtainable by liquid biopsy.

In some embodiments the prostate tumor sample contains compounds which are not naturally intermixed with the tissue in nature such as preservatives, anticoagulants, buffers, nutrients, antibiotics, or the like. In certain embodiments the prostate tumor sample has been exposed to and/or contains one or more fixatives. Fixatives that can be used with methods and compositions of the invention include formalin, glutaraldehyde, osmium tetraoxide, acetic acid, ethanol, acetone, picric acid, chloroform, potassium dichromate and mercuric chloride and/or stabilizing by microwave heating or freezing.

In some embodiments, the prostate tumor sample may be further processed for microscopic visualization, for example, by embedding preserved samples in paraffin, cutting the prostate tumor sample into smaller sections, and/or applying the prostate tumor sample to microscope slides. In one specific embodiment, the prostate tumor sample is a formalin-fixed paraffin embedded sample that has been section and affixed to a microscope slide. In another specific embodiment, the prostate tumor sample comprises circulating tumor cells affixed to a microscope slide.

### II. Binding Entities

As used herein, the term “binding entity” shall refer to any compound or composition that is capable of specifically binding to protein or a specific amino acid sequence or a specific structure within that protein. Examples include antibodies and antigen binding fragments thereof, as well as engineered specific binding structures, including ADNECTINs (scaffold based on 10^{th} FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, Calif.), dAbs (scaffold based on VH or VL antibody domain; GlaxoSmithKline PLC, Cambridge, UK), DARPins (scaffold based on Ankyrin repeat proteins; Molecular Partners AG, Zürich, CH), ANTICALINs (scaffold based on lipocalins; Pieris AG, Freising, DE), NANOBODYs (scaffold based on VHH (camelid Ig); Ablynx N/V, Ghent, BE), TRANS-BODYs (scaffold based on Transferrin; Pfizer Inc., New York, N.Y.), SMIPs (Emergent Biosolutions, Inc., Rockville, Md.), and TETRANECTINs (scaffold based on C-type lectin domain (CTLD), tetranectin; Borean Pharma A/S, Aarhus, DK). Descriptions of such engineered specific binding structures are reviewed by Wurch et al., Development of Novel Protein Scaffolds as Alternatives to Whole Antibodies for Imaging and Therapy: Status on Discovery Research and Clinical Validation, Current Pharmaceutical Biotechnology, Vol. 9, pp. 502-509 (2008).

As used herein, the phrase “specific binding,” “specifically binds to,” or “specific for” refers to measurable and reproducible interactions such as binding between a target and a binding entity, which is determinative of the presence of the target in the presence of a heterogeneous population of molecules including biological molecules. For example, a binding entity that specifically binds to a target is an antibody that binds this target with greater affinity, avidity, more readily, and/or with greater duration than it binds to other targets. In one embodiment, the extent of binding of a binding entity to an unrelated target is less than about 10% of the binding of the antibody to the target as measured, e.g., by a radioimmunoassay (RIA). In certain embodiments, a binding entity that specifically binds to a target has a dissociation constant (Kd) of ≦1 μM, ≦100 nM, ≦0 nM, ≦1 nM, or ≦0.1 nM. In another embodiment, specific binding can include, but does not require exclusive binding.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized antibodies, fully human antibodies, chimeric antibodies and camelized single domain antibodies.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

In one embodiment, the binding entity specific for the N-terminus is an antibody. These antibodies will generally be raised against at least a portion of the region within AR that is conserved between full length AR and C-terminal truncations thereof. An exemplary antibody specific for the N-terminus of AR is a Rabbit Anti-Human Androgen Receptor (AR) Monoclonal Antibody (Clone SP107) sold commercially by Spring Bioscience Inc. (Pleasanton, Calif.) (hereafter “SP107”). SP107 is a rabbit monoclonal IgG raised against a synthetic peptide derived from near the N-terminus of human androgen receptor. This antibody has been shown to bind specifically to the N-terminus of human AR. It is also contemplated that other N-terminal specific antibodies could be used in place of SP107. For example, antibodies that compete with SP107 for binding to human AR can be used in place of SP107. Other exemplary antibodies can be found at Zhang et al, Androgen Receptor Variants Occur Frequently in Castration Resistant Prostate Cancer Metastases, PLoS ONE Vol. 6, issue 11, e27970 (2011).

In another embodiment, the binding entity specific for the C-terminus is an antibody. An exemplary antibody specific for the C-terminus of AR is Rabbit anti-human Androgen Receptor (C-term) (SP242) monoclonal antibody sold commercially by Spring Bioscience Inc. (Pleasanton, Calif.) (hereafter “SP242”). SP242 is a rabbit monoclonal IgG raised against a synthetic peptide from the C-terminus of human androgen receptor protein. This antibody has been shown to bind specifically to the C-terminus of human AR, and is predicted by homology to bind to the C-terminus of dog, mouse, pig, rabbit, and rat AR. It is also contemplated that other C-terminal specific antibodies could be used in place of SP107. For example, antibodies that compete with SP242 for binding to human AR can be used in place of SP242.

As used herein, an “antibody that competes with” a reference antibody refers to an antibody that blocks binding of the reference antibody to its antigen in a competition assay by 50% or more, and conversely, the reference antibody blocks binding of the antibody to its antigen in a competition assay by 50% or more. In certain embodiments, such a competing antibody binds to the same epitope (e.g., a linear or a conformational epitope) that is bound by the reference antibody (e.g., SP242 or SP107). Detailed exemplary methods for mapping an epitope to which an antibody binds are provided in Morris “Epitope Mapping Protocols,” in Methods in Molecular Biology Vol. 66 (Humana Press, Totowa, N.J., 1996). In an exemplary competition assay, immobilized antigen (e.g. AR) is incubated in a solution comprising a first labeled antibody that binds to the antigen (e.g., SP242 or SP107) and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to antigen. See, e.g., Harlow et al. Antibodies: A Laboratory Manual. Ch. 14 (Cold Spring Harbor Laboratory, Cold Spring Harbor, N.Y., 1988).

### III. Labeling of the Prostate Tumor Sample

The prostate tumor sample is contacted with the first and second binding entities in a manner that results in deposition of a detectable label on the prostate tumor sample.

In some embodiments, the first and second binding entities may be modified to facilitate deposition of the label. For example, a detectable label may be conjugated directly to the binding entity. As another example, the first and second binding entity may be modified to include a moiety that is a part of a specific binding pair with a separate detectably labeled entity. Examples of such an arrangement include: biotinylated antibody/detectably-labeled streptavidin pairs; and haptenized antibody/detectably labeled anti-hapten antibody.

In other embodiments, the first and/or second binding entities are unmodified, and binding of the first and second binding entities is detected via use of an entity that specifically binds to the first and second binding entities. For example, where the first and second binding entities are unlabeled antibodies, the detectable label may be conjugated to at least a third binding entity capable of binding to the first and second binding entity, such as a secondary antibody. The term “secondary antibody” shall refer to an antibody that is specific for another antibody. Secondary antibodies can be specific for certain antibody classes, certain species of animal from which the antibody is derived, or both. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. Thus, for example, if the first binding entity is a rabbit IgG antibody and the second binding entity is a mouse IgG antibody, a third binding entity can be provided that is a secondary antibody specific for rabbit IgG and a fourth binding entity can be provided that is a secondary antibody specific for mouse IgG. As another example, the first and second binding entities can be antibodies of the same class and species (e.g. both rabbit IgG), in which case at least two ways can be used to deposit a detectable label. First, a “heat kill” method can be used. In a heat kill method, a single antigen-specific antibody is deposited onto a tissue, followed by a secondary antibody having a first detectable label. The sample is then heated in a microwave, which denatures the antigen-specific antibody and the secondary antibody and deposits the first detectable label on the tissue. The process is then repeated with a different antigen-specific antibody and a secondary antibody having a second detectable label that is different from the first detectable label. See generally Toth & Mezey, Simultaneous Visualization of Multiple Antigens With Tyramide Signal Amplification Using Antibodies From the Same Species, J. Histochemistry and Cytochemistry, Vol. 55, Issue 6, pp. 545-54 (2011), incorporated herein by reference in its entirety.

As used herein, the term “detectable label” refers to a molecule or material that can produce a detectable (such as visually, electronically or otherwise) signal that indicates the presence and/or concentration of the label in a sample. When associated with a binding entity (either directly or indirectly), the detectable label can be used to locate and/or quantify the target to which the binding entity is directed. Thereby, the presence and/or concentration of the target in a sample can be detected by detecting the signal produced by the detectable label. A detectable signal can be generated by any mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected through antibody-hapten binding interactions using additional detectably labeled antibody conjugates, and paramagnetic and magnetic molecules or materials. Particular examples of detectable labels include enzymes such as horseradish peroxidase, alkaline phosphatase, acid phosphatase, glucose oxidase, β-galactosidase or β-glucuronidase; fluorphores such as fluoresceins, luminophores, coumarins, BODIPY dyes, resorufins, and rhodamines (many additional examples of fluorescent molecules can be found in The Handbook—A Guide to Fluorescent Probes and Labeling Technologies, Molecular Probes, Eugene, Oreg.); nanoparticles such as quantum dots (obtained, for example, from QuantumDot Corp, Invitrogen Nanocrystal Technologies, Hayward, Calif.; see also, U.S. Pat. Nos. 6,815,064, 6,682,596 and 6,649,138, each of which patents is incorporated by reference herein); metal chelates such as DOTA and DPTA chelates of radioactive or paramagnetic metal ions like Gd 3+; and liposomes, for example, liposomes containing trapped fluorescent molecules. Where the detectable label includes an enzyme, a detectable substrate such as a chromogen, a fluorogenic compound, or a luminogenic compound can be used in combination with the enzyme to generate a detectable signal (A wide variety of such compounds are commercially available, for example, from Invitrogen Corporation, Eugene Oreg.). Particular examples of chromogenic compounds include diaminobenzidine (DAB), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet. Alternatively, an enzyme can be used in a metallographic detection scheme. Metallographic detection methods include using an enzyme such as alkaline phosphatase in combination with a water-soluble metal ion and a redox-inactive substrate of the enzyme. The substrate is converted to a redox-active agent by the enzyme, and the redox-active agent reduces the metal ion, causing it to form a detectable precipitate. (See, for example, co-pending U.S. patent application Ser. No. 11/015,646, filed Dec. 20, 2004, PCT Publication No. 2005/003777 and U.S. Patent Application Publication No. 2004/0265922; each of which is incorporated by reference herein). Metallographic detection methods include using an oxido-reductase enzyme (such as horseradish peroxidase) along with a water soluble metal ion, an oxidizing agent and a reducing agent, again to form a detectable precipitate. (See, for example, U.S. Pat. No. 6,670,113, which is incorporated by reference herein). Haptens are small molecules that are specifically bound by antibodies, although by themselves they will not elicit an immune response in an animal and must first be attached to a larger carrier molecule such as a protein to generate an immune response. Examples of haptens include di-nitrophenyl, biotin, digoxigenin, and fluorescein. Additional examples of oxazole, pyrazole, thiazole, nitroaryl, benzofuran, triperpene, urea, thiourea, rotenoid, coumarin and cyclolignan haptens are disclosed in U.S. Pat. No. 7,985,557, issued May 21, 2013, which is incorporated by reference herein. This is not an exhaustive review of all possible labeling schemes, and other useful labels and labelling schemes may be currently available or developed in the future.

The prostate tumor samples are labeled with the binding entities and detectable labels using standard histological or cytological techniques. In practicing the methods of this invention, staining procedures can be carried out by a person, such as a histo-technician in an anatomic pathology laboratory. Alternatively, the staining procedures can be carried out using automated systems, such as VENTANA BenchMark and VENTANA DISCOVERY automated stainers. In either case, staining procedures for use according to the methods of this invention are performed according to standard techniques and protocols well-established in the art.

### IV. Detection and Quantitation

The labeled prostate tumor samples are visualized microscopically and the degree of labeling of the first binding entity and the second binding entity is quantitated.

In one embodiment, detection and quantitation are performed manually. For example, slides can be visualized on a microscope by a trained reader (such as a pathologist). The trained reader can then visually score the staining. For example, a trained reader could classify the tissue sample on the basis of staining intensity for each of the first and second binding entities. For example, the trained reader could evaluate the slide and estimate the percentage of cells having strong staining, weak staining, and no staining for each of the markers. As another example, the trained reader could generate an H-score by, for example, examining a plurality of different fields at high magnification (such as 100×) and counting the number of cells at each of a plurality of intensity levels, each intensity level being assigned a numeric value with reducing value as intensity levels decrease. For example, 3 intensity levels can be assigned (e.g. 3=high intensity, 2=moderate intensity, and 1=low intensity) or 6 intensity levels can be assigned (e.g. 5=high intensity, 4=moderately high intensity; 3=moderate intensity; 2=moderately low intensity; 1=low intensity; and 0=no staining). An H-score can then be calculated according to the following formula:

Σ(i*%_{I}) Formula I

wherein I is the numerical value assigned to the intensity level and %_{I} is the percentage of cells staining at the designated intensity level.

In another embodiment, detection and quantitation are performed automatically by generating a digitized microscopic image of the prostate tumor sample and analyzing the digital image for the detectable labels. Numerous digital image scanners are known that can perform this process, including, for example, Aperio's SCANSCOPE XT device. The scanner will count the number of pixels staining at one of a plurality of predefined intensity levels and assign a numeric value to each of the predefined intensity levels. An H-score can then be calculated according to the following formula:

Σ(P*%_{P}) Formula II

wherein P is the numerical value assigned to the pixel intensity level and %_{P} is the percentage of pixels staining at the designated intensity level.

### V. Progression Tracking

In order to track progression of a prostate cancer, a prostate tumor sample from the same patient is collected at least at two different time points. Staining levels using the first and the second binding entities are determined and a binding score is calculated for each of the first and second binding entities in each of the samples. A ratio is calculated for each sample according to the following formula:

B₁/B₂ Formula III

wherein B₁ is the binding score of the first binding entity to the prostate tumor sample, and B₂ is the binding score of the second binding entity to the prostate tumor sample. The ratios are compared to one another and, if the ratios increase from earlier to later time points, the tumor is considered to be progressing. If the ratios do not significantly change, the tumor is considered to be stable. If the ratios decrease from earlier to later time points, the tumor is considered to be regressing.

### VI. Prognosis and Predicting Therapeutic Resistance

In order to prognose a cancer, one will need to collect a prostate tumor samples from the patient and calculate a binding score for each of the first and second binding entities. A ratio will then be calculated for the sample according to Formula III. The ratio is then compared to a reference ratio.

The reference ratio could be a baseline ratio generated from the same patient at an earlier time point, in which case an increase in the ratio compared to the reference ratio indicates a poor prognosis.

Alternatively, the reference ratio could be a “cutoff” ratio calculated from a representative patient population, wherein ratios falling above the cutoff significantly correlate with a poor prognosis, whereas ratios falling below the cutoff significantly correlate with a good prognosis. It is possible that the “reference ratio” may be represented as a range of ratios, such that ratios falling within the range of ratios do not have a statistically significant correlation with either a poor prognosis or a good prognosis. As used herein, a “good prognosis” and a “poor prognosis” may represent: (1) an indication of whether or not the prostate cancer is likely to progress within a defined period of time; (2) an indication of whether or not the patient is likely to respond to a treatment course (such as administration of an AR-targeted therapeutic agent or a chemotherapeutic agent); (3) an indication of the mortality rate over a defined period of time.

As used herein, the phrase “AR-directed therapeutic” shall refer to any therapeutic course that attempts to interfere with AR-mediated signaling. Types of AR-directed therapeutics include: AR antagonists, which act by preventing interaction between AR androgens; and inhibitors of androgen synthesis, which act by preventing androgens from being produced. Specific examples of AR-directed therapeutics include abiraterone, enzalutamide, Orteronel, Galeterone, ARN-509, ODM-201, AZD3514, EZN-4176, and BMS-641988.

As used herein, the phrase “chemotherapeutic agent” encompasses any drug used to treat prostate cancer other than an AR-directed therapeutic. Specific examples include taxanes, such as paclitaxel and docetaxel.

### VII. Selection of Treatment Course

In order to select a treatment course, a prostate tumor sample is collected from the patient and a binding score is calculated. A ratio will then be calculated for the sample according to Formula III and compared to a reference ratio. If the ratio falls above the reference ratio, then an aggressive treatment course is chosen. If the ratio falls below the reference ratio, when a conservative treatment course is chosen.

As used herein, the term “aggressive treatment course” refers to invasive or particularly risky treatments, such as surgical removal of the tumor and/or prostate, castration, and/or radiation therapy.

As used herein, the term “conservative treatment course” includes any treatment course that is not an aggressive treatment course, including surveillance or administration of an AR-directed therapeutic agent and/or a chemotherapeutic agent.

The reference ratio could be a baseline ratio generated from the same patient at an earlier time point, in which case a significant increase in the ratio compared to the reference ratio suggests an aggressive treatment course, whereas a stable or reduction compared to the reference ratio indicates a conservative treatment course.

Alternatively, the reference ratio could be a “cutoff” ratio calculated from a representative patient population, wherein ratios falling above the cutoff significantly correlate with a poor prognosis, whereas ratios falling below the cutoff significantly correlate with a good prognosis. It is possible that the “reference ratio” may be represented as a range of ratios, such that ratios falling within the range of ratios do not have a statistically significant correlation with either a poor prognosis or a good prognosis. As used herein, a “good prognosis” and a “poor prognosis” may represent: (1) an indication of whether or not the prostate cancer is likely to progress within a defined period of time; (2) an indication of whether or not the patient is likely to respond to a treatment course (such as administration of an AR-targeted therapeutic agent or a chemotherapeutic agent); (3) an indication of the mortality rate over a defined period of time.

### VIII. Monitoring a Treatment Course

In order to monitor a treatment course, a prostate tumor sample is collected from the patient at or near to the beginning of the treatment course and then again at least at one time point during or after the treatment course and a binding score is calculated for each prostate tumor sample. A ratio is calculated for the samples according to Formula III, and ratios are compared to one another. If the ratio increases during the course of the treatment, the prostate cancer is considered to be progressing and/or resistant to the treatment course and the treatment course is modified or halted, and/or a further treatment course is started. For example, the initial treatment course may comprises surveillance or administration of an AR-directed therapeutic agent and/or a chemotherapeutic agent. If the ratio remains the same or goes down, the initial treatment course is continued. If the ratio increases, a further treatment course comprising surgical removal of the tumor and/or prostate, castration, and/or radiation therapy. The monitoring may then continue after the further treatment and the further treatment is halted if the ratio continues to increase during the course of the further treatment.

### EXAMPLES

### I. Materials and Methods

A. Antibodies

The present study uses two primary rabbit monoclonal antibodies targeting the N-terminus and the C-terminus of human AR. Anti-Androgen Receptor (SP107) Rabbit Monoclonal Primary Antibody (Ventana Medical Systems, Inc., cat #760-4605) is used as the antibody targeting the N-terminus. Androgen Receptor (AR) (C-term) (SP242) rabbit monoclonal primary antibody (Spring Bioscience, cat# M5424) is used as the antibody targeting the C-terminus.

B. Control Cell Lines

LNCaP, VCaP, CWR22v1, PC3 prostate cell lines were analyzed by RT-PCR and Western blot to confirm their AR-FL and AR-v7 status. In addition, M12AR and M12 AR-v7 (University of Washington) were stained by IHC.

C. Clinical Samples

FFPE, tissue microarrays (TMA) of prostatic tissues (University of Washington), representing the progression of prostate cancer (80 primary naïve prostate cancer cores and 160 CRPC soft and bone metastasis cores) were stained with both antibodies. Selected images from the TMAs were available for image analysis (2 cases of primary tumors, and 4 metastatic tumors).

D. RT-PCR Assays

Primers were designed and synthesized for AR-FL, AR-v7 and house-keeping gene RPL13A RNA extraction was carried out with Qiagen RNEASY mini kit with on column DNase I treatment, and post extraction clean up with Qiagen gDNA. Invitrogen SUPERSCRIPT III first-strand synthesis system was used for cDNA library preparation. RNA negative (no RNA) control and gDNA contamination (no RT) control were run side by side during assay set-up on cell line samples. PCR was performed and the PCR products were analyzed on a 1.5% agarose gel (GENEWIZ, South Plainfield, N.J.).

E. Western Blot

Cell lysates were prepared using COMPLETE LYSIS-M kit (Roche Life Science, Branford, Conn.). 10 μg of total protein was loaded to each lane. Lysates were then immunoblotted with specified antibodies. Antibody working concentration was 4.4 μg/ml for SP107, 24 μg/ml for SP242.

F. IHC Performance Characterization Method

IHC staining was performed on consecutive sections of FFPE control cell lines and clinical samples referenced above in both Tucson, Ariz. and Seattle, Wash. on a VENTANA Discovery XT instrument. The antigen-antibody complexes were detected with an OPTIVIEW DAB kit (Ventana Medical Systems Inc., Tucson, Ariz.).

G. Scoring/Image Analysis (Manual and Digital H-Score):

Slides were scored visually using a categorical method (no stain, faint, and intense). Slides were also scanned by Aperio's SCANSCOPE XT device. Areas representing the entire tissue or cell pellet were annotated for image analysis. Analysis was performed using a positive pixel elective algorithm. “Heat Map” is generated based upon staining intensity, according to Weak (W), Moderate (M) and Strong (S) DAB stained pixels. A data analysis step was performed to obtain the percentage of W, M and S pixels/total pixels. Then an equivalent H-score was used to represent the staining by using the following formula: (1*% pixels staining at 1(W)*100+2*% pixels staining at 2 (M)*100+3*% pixels staining at 3(S)]=digital H-Score ranging from 0-300).

### II. Results

### Validation of Cell Lines and Antibodies

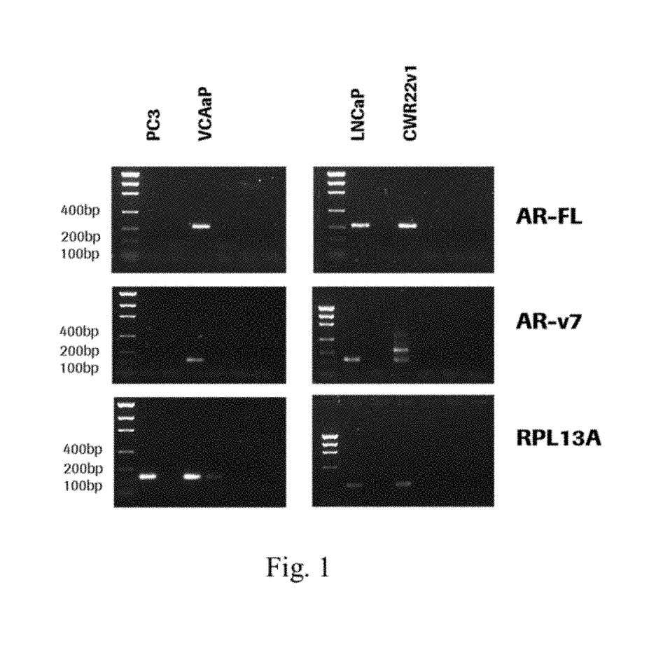

RT-PCR was used to ensure that the cell lines used express the expected AR variant. As can be seen at FIG. 1, the presence of the full length AR-FL and AR-v7 was confirmed in LNCaP, VCaP and CWR22v1 cell lines (positive controls), while neither AR-FL nor AR-v7 was detected in PC3 cell line (negative control). See FIG. 1.

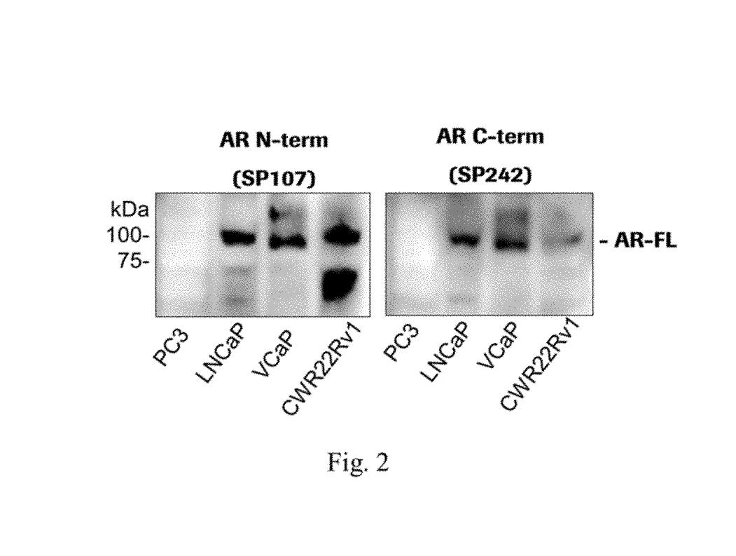

Antibody specificity was validated in the validated cell lines. AR-FL could be consistently detected by Western blot in both SP107 and SP242 antibodies in AR positive cell lines including LNCaP, VCaP and CWR22Rv1, while AR-FL was not detectable by either antibodies in AR negative cell line, PC3. See FIG. 2.

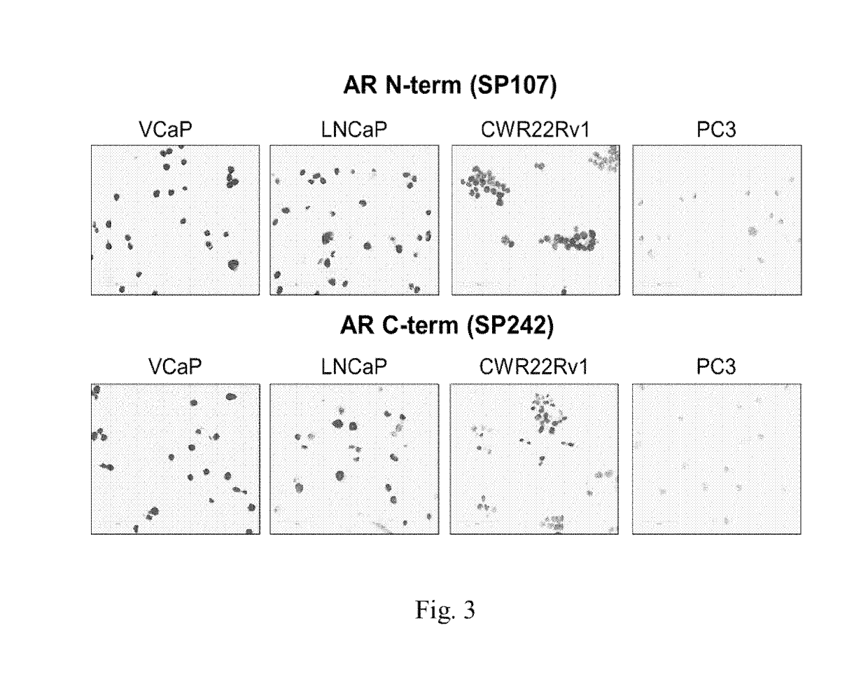

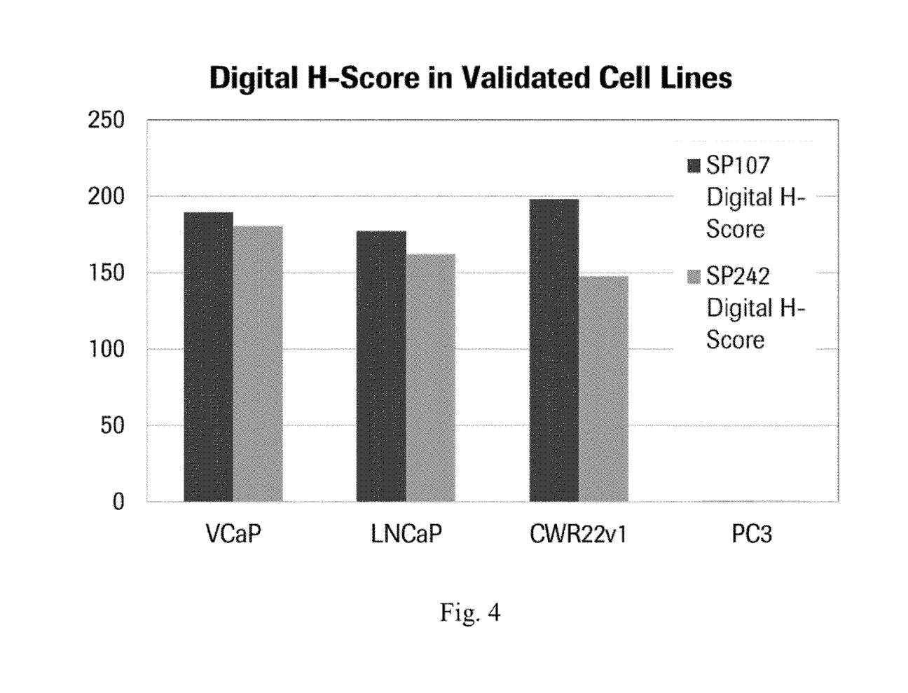

The validated cell lines were stained immunohistochemically using the SP107 and SP242 cell lines. As can be seen at FIG. 3, LNCaP, VCaP and CWR22Rv1 were positively stained with each of SP107 and SP242, while staining was not detectable in PC3. H-scores were calculated, the results of which are demonstrated at FIG. 4 and Table 1:

TABLE 1

| | SP107 | SP242 | SP107:SP242 |
|---|---|---|---|
| | Digital H-score | Digital H-score | Ratio |
| VCaP | 189.50 | 180.30 | 1.05 |
| LNCaP | 177.10 | 161.80 | 1.10 |
| CWR22v1 | 198.20 | 147.80 | 1.34 |
| PC3 | 0.20 | 0.40 | 0.53 |

### Utility in Clinical Samples

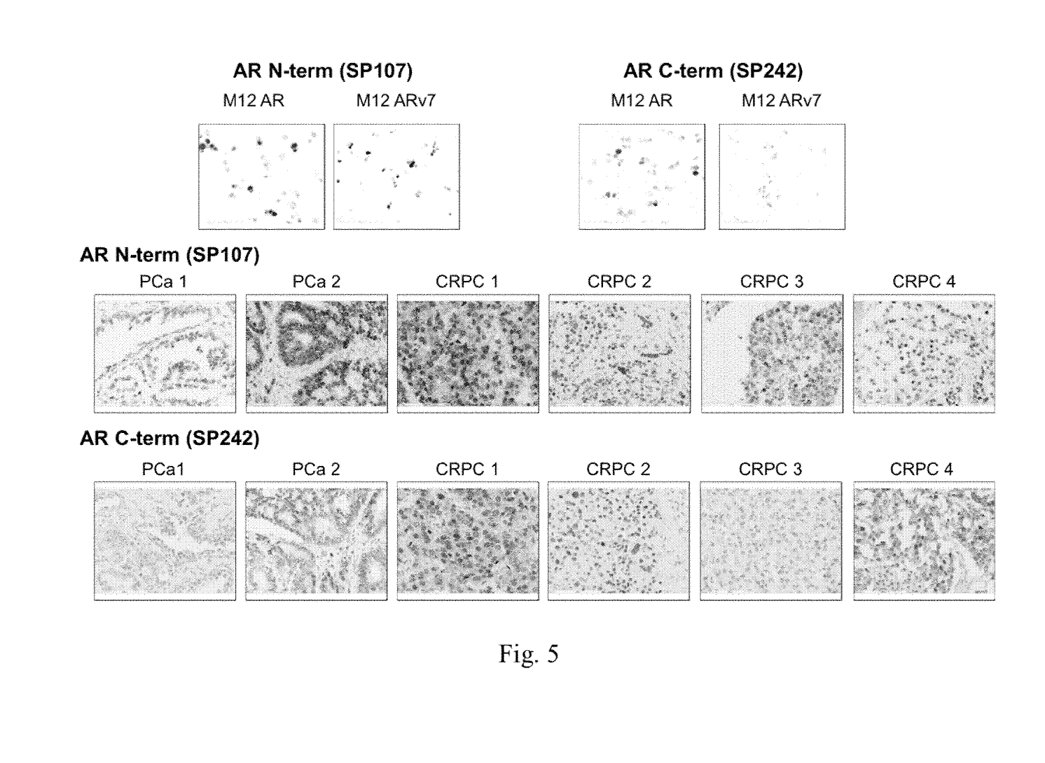

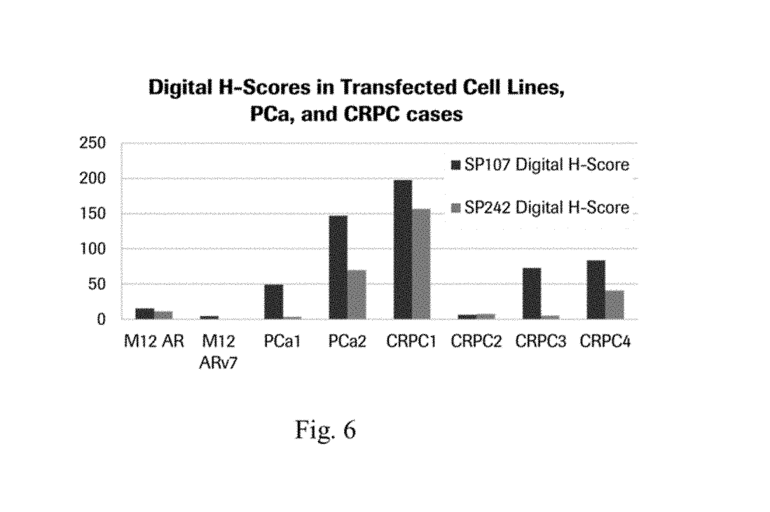

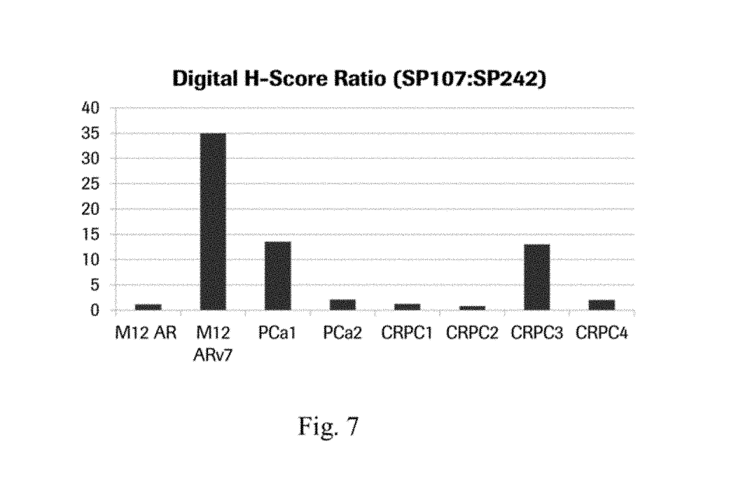

SP107 and SP242 were used for IHC staining on cell lines transfected with full length AR (M12 AR) or AR C-terminal splice variant ARv7 (M12 ARv7), primary prostate tumors (PCa), and metastatic tumors (CRPC). Representative images are shown at FIG. 5 (40× magnification). Digital H-scores (Table 2 and FIG. 6) and manual categorical scoring (table 3) were calculated for on transfected cell lines, PCa, and CRPC for both SP107 and SP242 and their comparative ratio. Comparative ratios were calculated (FIG. 7).

TABLE 2

| | SP107 | SP242 | SP107:SP242 |
|---|---|---|---|
| | Digital H-score | Digital H-score | Ratio |
| M12 AR | 15.9 | 11.7 | 1.15 |
| M12 ARv7 | 4.8 | 0.1 | 34.98 |
| PCa1 | 49.19 | 3.63 | 13.56 |
| PCa2 | 146.84 | 69.89 | 2.10 |
| CRPC1 | 197.43 | 156.77 | 1.26 |
| CRPC2 | 6.56 | 7.73 | 0.85 |
| CRPC3 | 73.06 | 5.62 | 12.99 |
| CRPC4 | 83.99 | 41.20 | 2.04 |

TABLE 3

| | SP107 Visual Score | | | SP242 Visual Score | | |
|---|---|---|---|---|---|---|
| | % no | % | % | % no | % | % |
| | stain | faint | intense | stain | faint | intense |
| M12 AR | 50 | 0 | 50 | 60 | 5 | 35 |
| M12 ARv7 | 55 | 5 | 40 | 100 | 0 | 0 |
| PCa1 | 5 | 10 | 85 | 95 | 5 | 0 |
| PCa2 | 0 | 5 | 95 | 15 | 25 | 60 |
| CRPC1 | 5 | 20 | 75 | 5 | 10 | 85 |
| CRPC2 | 10 | 30 | 60 | 100 | 0 | 0 |

1. A method of tracking progression of a prostate cancer in a patient, the method comprising:
(a) microscopically detecting binding of a first binding entity and a second binding entity to a prostate tumor sample from the patient, wherein the first binding entity binds specifically to the N-terminal domain of androgen receptor (AR) and the second binding entity binds specifically to the C-terminal ligand binding domain of AR;
(b) calculating a ratio of B₁/B₂, wherein:
B₁ is binding of the first binding entity to the prostate tumor sample, and
B₂ is binding of the second binding entity to the prostate tumor sample; and
(c) comparing the ratio of (b) to a reference ratio calculated according to (b) for a sample of the same tumor taken from the patient at an earlier time point;
wherein an increase in the ratio compared to the reference ratio indicates progression of the prostate cancer.

2. A method of prognosing a prostate cancer in a patient, the method comprising
(a) microscopically detecting binding of a first binding entity and a second binding entity to a prostate tumor sample from the patient, wherein the first binding entity binds specifically to the N-terminal domain of androgen receptor (AR) and the second binding entity binds specifically to the C-terminal ligand binding domain of AR;
(b) calculating a ratio of B₁/B₂, wherein:
B₁ is binding of the first binding entity to the prostate tumor sample, and
B₂ is binding of the second binding entity to the prostate tumor sample; and
(c) comparing the ratio of (b) to a reference ratio;
wherein a higher ratio of (b) compared to the reference ratio indicates a poor prognosis.

3. The method of claim 2, wherein the reference ratio is a ratio calculated according to (b) for a sample of the same tumor taken from the patient at an earlier time point.

4. The method of claim 2, wherein the reference ratio is a ratio calculated according to (b) for a representative number of prostate tumors taken from a general patient population.

5. The method of claim 4, wherein the reference ratio is a cutoff separating patients expected to respond to an AR-targeted therapeutic agent or a chemotherapeutic agent from patients expected to be resistant to the same AR-targeted therapeutic agent and/or chemotherapeutic agent.

6. The method of claim 5, wherein the AR-directed therapeutic agent is an AR antagonist.

7. The method of claim 5, wherein the AR-directed therapeutic is an inhibitor of androgen synthesis.

8. The method of claim 5, wherein the AR-targeted therapeutic agent is selected from the group consisting of abiraterone, enzalutamide, Orteronel, Galeterone, ARN-509, ODM-201, AZD3514, EZN-4176, and BMS-641988.

9. The method of claim 5, wherein the AR-targeted therapeutic agent is abiraterone or enzulutamide.

10. The method of claim 5, wherein the chemotherapeutic agent is a taxane.

11. The method of claim 10, wherein the taxane is paclitaxel or docetaxel.

12. A method of predicting resistance of a patient having prostate cancer to an androgen receptor (AR)-targeted therapeutic agent and/or a chemotherapeutic agent, the method comprising:
(a) microscopically detecting binding of a first binding entity and a second binding entity to a prostate tumor sample from the patient, wherein the first binding entity binds specifically to the N-terminal domain of androgen receptor and the second binding entity binds specifically to the C-terminal ligand binding domain of androgen receptor;
(b) calculating a ratio of B₁/B₂, wherein:
B₁ is binding of the first binding entity to the prostate tumor sample, and
B₂ is binding of the second binding entity to the prostate tumor sample; and
(c) comparing the ratio of (b) to a reference ratio;
wherein a higher ratio according to (b) as compared to the reference ratio indicates that the prostate cancer is unlikely to respond to the AR-targeted therapeutic agent and/or a chemotherapeutic agent.

13. The method of claim 12, wherein the reference ratio is a ratio calculated according to (b) for a sample of the same tumor taken from the patient at an earlier time point.

14. The method of claim 12, wherein the reference ratio is a ratio calculated according to (b) for a representative number of prostate tumors taken from a general patient population.

15. The method of claim 12, wherein the AR-directed therapeutic agent is an AR antagonist.

16. The method of claim 12, wherein the AR-directed therapeutic is an inhibitor of androgen synthesis.

17. The method of claim 12, wherein the AR-targeted therapeutic agent is selected from the group consisting of abiraterone, enzalutamide, Orteronel, Galeterone, ARN-509, ODM-201, AZD3514, EZN-4176, and BMS-641988.

18. The method of claim 12, wherein the AR-targeted therapeutic agent is abiraterone or enzulutamide.

19. The method of claim 12, wherein the chemotherapeutic agent is a taxane.

20. The method of claim 19, wherein the taxane is paclitaxel or docetaxel.

21. A method of selecting a treatment course for a patient having prostate cancer, the method comprising:
(a) characterizing the prostate cancer for the presence of C-terminal deletion splice variants of androgen receptor by:
(a1) microscopically detecting binding of a first binding entity and a second binding entity to a prostate tumor sample from the patient, wherein the first binding entity binds specifically to the N-terminal domain of androgen receptor and the second binding entity binds specifically to the C-terminal ligand binding domain of androgen receptor;
(a2) calculating a ratio of B₁/B₂, wherein:
B₁ is binding of the first binding entity to the prostate tumor sample, and
B₂ is binding of the second binding entity to the prostate tumor sample, and
(a3) comparing the ratio of (a2) to a reference ratio; and
(b) selecting the treatment course based on (a), wherein:
(b1) an aggressive treatment course is selected when the ratio of (a2) is greater than the reference ratio; and
(b2) a conservative treatment course is selected when the ratio of (a2) is less than the reference ratio.

22. The method of claim 21, wherein the aggressive treatment course comprises surgical removal of the tumor and/or prostate, castration, and/or radiation therapy.

23. The method of claim 21, wherein the conservative treatment course comprises surveillance or administration of an AR-directed therapeutic agent and/or a chemotherapeutic agent.

24. The method of claim 23, wherein the AR-directed therapeutic agent is an AR antagonist.

25. The method of claim 23, wherein the AR-directed therapeutic is an inhibitor of androgen synthesis.

26. The method of claim 23, wherein the AR-targeted therapeutic agent is selected from the group consisting of abiraterone, enzalutamide, Orteronel, Galeterone, ARN-509, ODM-201, AZD3514, EZN-4176, and BMS-641988.

27. The method of claim 23, wherein the AR-targeted therapeutic agent is abiraterone or enzulutamide.

28. The method of claim 23, wherein the chemotherapeutic agent is a taxane.

29. The method of claim 28, wherein the taxane is paclitaxel or docetaxel.

30. A method of monitoring a treatment course of a prostate cancer in a patient, the method comprising:
(a) microscopically detecting binding of a first binding entity and a second binding entity to a prostate tumor sample from the patient, wherein the first binding entity binds specifically to the N-terminal domain of androgen receptor (AR) and the second binding entity binds specifically to the C-terminal ligand binding domain of AR;
(b) calculating a ratio of ratio of B₁/B₂ at a plurality of time points during a treatment course, wherein:
B₁ is binding of the first binding entity to the prostate tumor sample, and
B₂ is binding of the second binding entity to the prostate tumor sample; and
(c) comparing the ratios of (b);
wherein an increase in the ratio during the course of treatment indicates progression of the prostate cancer and/or resistance to the treatment course.

31. The method of claim 30, wherein the treatment course comprises surveillance or administration of an AR-directed therapeutic agent and/or a chemotherapeutic agent.

32. The method of claim 31, wherein the AR-directed therapeutic agent is an AR antagonist.

33. The method of claim 31, wherein the AR-directed therapeutic is an inhibitor of androgen synthesis.

34. The method of claim 31, wherein the AR-targeted therapeutic agent is selected from the group consisting of abiraterone, enzalutamide, Orteronel, Galeterone, ARN-509, ODM-201, AZD3514, EZN-4176, and BMS-641988.

35. The method of claim 31, wherein the AR-targeted therapeutic agent is abiraterone or enzulutamide.

36. The method of claim 31, wherein the chemotherapeutic agent is a taxane.

37. The method of claim 36, wherein the taxane is paclitaxel or docetaxel.

38. The method of claim 30, wherein a further treatment course is selected when the ratio increases, the further treatment course comprising surgical removal of the tumor and/or prostate, castration, and/or radiation therapy.

39. The method of claim 38, further comprising repeating (a) and (b) during the course of the further treatment, wherein further treatment is halted if the ratio increases during the course of the further treatment.

40. The method of claim 21, wherein binding of the first and second binding agent is detected immunohistochemically.

41. The method of claim 40, wherein B₁ and B₂ are measures of signal intensity.

42. The method of claim 41, wherein an H-score is calculated for the first and second binding agents and the ratio is a ratio of the H-scores.

43. The method of claim 42, wherein the H-scores are calculated on the basis of nuclear and cytosolic staining of the first and second binding agents.

44. The method of claim 42, wherein the H-scores are calculated on the basis of staining intensity of the first and second binding agents.

45. The method of claim 42, wherein the H-scores are automatically calculated using a digital image of the sample of tumor tissue.

46. The method of claim 45, wherein the H-scores are calculated using a positive pixel elective algorithm.

47. The method of claim 30, wherein the first binding entity and/or second binding entity is an antibody.

48. The method of claim 47, wherein the first binding entity is monoclonal antibody SP107.

49. The method of claim 47, wherein the first binding entity is an antibody that competes with SP107 for binding to androgen receptor.

50. The method of claim 47, wherein the second binding entity is monoclonal antibody SP242.

51. The method of claim 47, wherein the second binding entity is an antibody that competes with SP242 for binding to androgen receptor.

52. The method of claim 30, wherein the first binding entity and the second binding entity are labeled with a chromogenic agent or a fluorescent agent.

53. The method of claim 52, wherein the chromogenic agent or the fluorescent agent is attached to a third binding entity capable of specifically binding to the first binding entity and a fourth binding entity capable of binding to the second binding entity.

54. The method of claim 53, wherein the first binding entity and the second binding entity comprise a non-endogenous hapten and the third binding entity and the fourth binding entity are antibodies or fragments thereof capable of specifically binding to the non-endogenous hapten.

55. A system comprising:
(a) an analytical imaging analysis system comprising:
a processor; and
a memory coupled to the processor, the memory to store computer-executable instructions that, when executed by the processor, cause the processor to perform operations comprising the method of claim 21.

56. The system of claim 55, further comprising:
(b) an analytical imaging hardware system adapted to capture a digitized image of the prostate tumor sample and to communicate the digitized image to the analytical imaging analysis system.
